(19) United States
(12) Patent Application Publication
(10) Pub. No.: US 2016/0011185 A1
(43) Pub. Date: Jan. 14, 2016

(54) MULTI-ANALYTE ASSAY

(71) Applicants: Gregory M. LAWRENCE, West Boylston, MA (US); Lisa SHINEFELD, (US); VERAX BIOMEDICAL INCORPORATED, Marlbororugh, MA (US)

(72) Inventors: Gregory M. Lawrence, West Boylston, MA (US); Lisa Shinefeld, Lexington, MA (US)

(21) Appl. No.: 14/772,657

(22) PCT Filed: Mar. 4, 2014
(86) PCT No.: PCT/US14/20430

§ 371 Date: Sep. 3, 2015

Related U.S. Application Data
(60) Provisional application No. 61/772,521, filed on Mar. 4, 2013.
(60) Provisional application No. 61/772,518, filed on Mar. 4, 2013.

(51) Int. Cl. G01N 33/543

(52) U.S. Cl. CPC G01N 33/54366

(57) ABSTRACT

The present invention is directed to devices and methods using pan-generic antibodies to detect bacteria in a sample. The invention relates to binding assays, especially immunoassays, utilizing a multivalent binding agent immobilized on a particle. The invention also relates to the surprising discovery that increasing the size of the particles improves the sensitivity of the screen. The invention provides, inter alia, a lateral flow device for detecting bacteria in a sample, the device comprising a flow path for the sample and further comprising a pan-generic binding agent specific for other or more bacterial antigens, wherein the pan-generic binding agent is immobilized via a linker on a population of particularly-sized detectable particles; and a capture binding agent that captures the population of particles bound to bacterial antigens, wherein the capture binding agent is immobilized on the flow path, and wherein the population of detectable particles are disposed along the flow path such that the sample contacts the population of detectable particles before contacting the capture binding agent.

### CROSS-REFERENCE TO RELATED APPLICATIONS

This application claims the benefit of U.S. Provisional Patent Application Nos. 61/772,518 and 61/772,521, filed Mar. 4, 2013. The disclosure of each of those applications is incorporated by reference herein in its entirety.

### FIELD OF THE INVENTION

The invention relates to binding assays, especially immunoassays, utilizing a multivalent binding agent immobilized on a particle. The invention also relates to the surprising discovery that increasing the size of the particles improves the sensitivity of the screen.

### BACKGROUND OF THE INVENTION

Testing liquid samples for bacterial contamination is a critical component in a wide variety of fields, such as medicine (e.g., testing blood samples for transfusion), environmental safety (e.g., testing water samples for human use), and food safety (e.g., testing food and beverage samples for consumption). The importance of bacterial testing necessitates tests that are rapid, sensitive, and broadly specific enough to detect a wide variety of bacterial species and genera. Practical limitations, such as the amount of a detection reagent (e.g., a bacterial antigen-binding antibody) or the visibility of a “positive” result in an assay may control the ability of current bacterial testing methods to meet these requirements. Thus, there is a need for improved reagents, devices and methods for rapidly, broadly and sensitively detecting bacteria.

### BRIEF SUMMARY OF THE INVENTION

In a first aspect, the invention provides a lateral flow device for detecting bacteria in a sample, the device comprising a flow path for the sample and further comprising a pan-generic binding agent specific for one or more bacterial antigens, wherein the pan-generic binding agent is immobilized via a linker on a population of particularly-sized detectable particles; and a capture binding agent that captures the population of particles bound to bacterial antigens, wherein the capture binding agent is immobilized on the flow path, and wherein the population of detectable particles are disposed along the flow path such that the sample contacts the population of detectable particles before contacting the capture binding agent. In some embodiments, the detectable particle is a chemiluminescent, a luminescent, a fluorescent, a magnetic or a colored particle. For convenience, the term “colored particle” will be used but the invention contemplates embodiments using other forms of detectable particles. In embodiments utilizing a colored particle, the particle may be a gold, silver, or platinum particle. In some embodiments, the particle is from about 60 to about 120 nm in diameter. In some embodiments the particle is about 80 nm in diameter.

In some embodiments, the pan-generic binding agent specifically binds a Gram-positive bacterial antigen. In some such embodiments, the pan-generic polyclonal antibody binds lipoteichoic acid (LTA). In some embodiments, the pan-generic binding agent specifically binds a Gram-negative bacterial antigen. In some such embodiments, the pan-generic polyclonal antibody binds a bacterial lipopolysaccharide structure (LPS). In some embodiments, at least one pan-generic binding agent specifically binds a Gram-positive bacterial antigen and at least one pan-generic binding agent specifically binds a Gram-negative bacterial antigen. In some embodiments, the pan-generic binding agent is capable of binding three or more genera of bacteria. In some embodiments, the linker is protein A, protein G, or protein L.

In some embodiments, the pan-generic binding agent is an antibody. In some embodiments the pan-generic binding agent comprises two or more pan-generic antibodies, wherein each pan-generic antibody specifically binds one or more bacterial antigens. In various embodiments, each pan-generic antibody is immobilized on a separate subpopulation or on the same subpopulation of colored particles. In some embodiments, the pan-generic binding agent can be combined with one or more binding agents that is not pan-generic. For example, a binding agent that is not pan-generic may bind one or more species or strains of bacteria but not to multiple genera.

In some embodiments, the one or more population of particles comprises a first population of particles and a second population of particles. In some embodiments, the first population of particles has one or more binding agents having a specificity for one or more first populations of antigens and the second population of particles has one or more binding agent having a specificity for one or more second populations of antigens, wherein the first one or more populations of antigens and the second one or more populations of antigens are different from each other.

In some embodiments, the pan-generic antibody is selected from a polyclonal antibody, a monoclonal antibody and a combination of polyclonal and monoclonal antibodies. In some embodiments, the pan-generic antibody is polyclonal and binds a plurality of bacterial antigens. In some embodiments, the pan-generic antibody is polyclonal and binds a plurality of Gram-positive bacterial antigens. In some embodiments, the pan-generic antibody is polyclonal and binds a plurality of Gram-negative bacterial antigens. In some embodiments, the pan-generic antibody is polyclonal and binds a plurality of Gram-negative bacterial antigens and Gram-positive bacterial antigens. In some embodiments, at least one pan-generic antibody is a monoclonal pan-generic antibody and at least one pan-generic antibody is a polyclonal pan-generic antibody.

In some embodiments, the pan-generic antibody specifically binds a Gram-positive bacterial antigen. In some embodiments, the pan-generic antibody specifically binds a Gram-negative bacterial antigen. In some embodiments, at least one pan-generic antibody specifically binds a Gram-positive bacterial antigen and at least one pan-generic antibody specifically binds a Gram-negative bacterial antigen. In some embodiments, the pan-generic antibody is capable of binding three or more genera of bacteria.

In some embodiments, the device comprises at least three pan-generic binding agents that specifically bind Gram-positive bacterial antigens, each pan-generic binding agent immobilized on a separate subpopulation of colored particles; and at least three pan-generic binding agents that specifically bind Gram-negative bacterial antigens, each pan-generic binding agent immobilized on a separate subpopulation of colored particles. In some embodiments, at least one pan-generic binding agent is an antibody. In some embodiments, at least one pan-generic antibody is a monoclonal antibody. In some embodiments, the subpopulations of particles are of different sizes. In some embodiments, the particles are gold, silver, or platinum. In some embodiments, at least some of the particles are from about 20 nm to about 120 nm in diameter. In some embodiments, at least some of the particles are gold particles from about 20 nm to about 120 nm in diameter. In some embodiments, at least one particle population (e.g., a gold particle population) comprises a 80 nm particle. In some embodiments, at least one particle population (e.g., a gold particle population) comprises a 40 nm particle.

In some embodiments, the capture binding agent is a pan-generic antibody that specifically binds a bacterial antigen. In some embodiments, the capture binding agent is the same as the pan-generic binding agent. In some embodiments, the capture antibody is immobilized in one or more locations on the sample flow path. In some embodiments, the sample flow path is an absorbent membrane. In some embodiments, the absorbent membrane is nitrocellulose.

In some embodiments the colored particles are dried within a solid support surface disposed above the absorbent membrane and in contact with the upper surface of the membrane.

In a second aspect, the invention provides a method for detecting the presence or absence of bacteria in a sample, comprising contacting the sample with a pan-generic binding agent specific for a bacterial antigen, wherein the pan-generic binding agent is immobilized via a linker on a colored particle, and wherein the sample is contacted with the pan-generic binding agent under conditions that permit binding between the pan-generic binding agent and a bacterial antigen to form a binding agent-bacterial antigen complex, and further comprising contacting an immobilized capture binding agent specific to a bacterial antigen with the colored particles under conditions that permit binding between the immobilized capture binding agent and the particle-pan-generic binding agent-bacterial antigen complex, wherein capture of the colored particle with the pan-generic binding agent by the capture binding agent indicates the presence of bacteria in the sample. In some embodiments, a small amount of soluble pan-generic binding agent is added to the sample before the assay is performed. Such small amount is an amount sufficient to improve the signal of the system.

In some embodiments, the method comprises contacting a device according to the first aspect of the invention with a sample under conditions that permit binding of the capture antibody to the colored particle with the pan-generic antibody, wherein capture of the particle by the capture antibody indicates the presence of bacteria in the sample.

In some embodiments, the sample is pre-treated.

According to the invention, a sample can be any liquid sample that is suspected of containing bacteria. In some embodiments, the sample is a biological fluid, including urine, sputum, spinal fluid, ascites, blood and blood products. In some embodiments, the sample is any liquid sample that is suspected of containing bacteria. In some embodiments, the sample is blood or a blood product. In some embodiments the blood or blood product is selected from the group consisting of: whole blood, leukocytes, hematopoietic stem cells, platelets, red blood cells, plasma, bone marrow and serum.

In some embodiments, the blood or a blood product such as platelets is from a donor for transfusion to a recipient. In some embodiments the sample is a dialysis sample. In some embodiments, the dialysis sample is selected from hemodialysis fluid and peritoneal dialysis fluid. In some embodiments, the sample is a sample of fluid in which a tissue such as a tissue from a donor for transplanting to a recipient has been stored. In some embodiments, the tissue is selected from the group consisting of: blood cell cultures, stem cell cultures, skin and bone and cartilage graft materials. In some embodiments, the sample is a sample from lung, bronchoalvealor, peritoneal, or arthroscopic lavage. In some embodiments, the samples are environmental samples such as water and soil. In some embodiments, the samples are foods or beverages. Those of skill in the art will recognize that in cases where the sample source is in solid form, such as soil or solid foods, the sample may be liquid that is extracted from the solid form or liquid that has been in contact with the solid form. In some embodiments, the sample is a biological sample, for example, urine, tears, sputum or cerebrospinal fluid.

In a third aspect, the invention provides a reagent for use in a binding assay comprising a particle selected from a gold particle, a silver particle and a platinum particle, wherein the particle size is from about 20 nm to about 120 nm, and wherein the particle is bound via a linker to a multi-specific pan-generic binding agent. In some embodiments, the particle size is about 80 nm. In some embodiments, the linker is selected from protein A, protein G and protein L. In some embodiments, the linker is protein A. In some embodiments, the particle is gold.

In a fourth aspect, the invention provides a method for detecting a substance in a sample comprising mixing the sample with a reagent according to the third aspect of the invention, wherein binding of the substance to the reagent creates a detectable complex; and detecting the complex.

In a fifth aspect, the invention provides a method for making a particle that has a very high surface density of linker, and thus a very high surface density of binding agent. The method according to this aspect comprises incubating colored particles with a high concentration of linker.

In a sixth aspect, the invention provides a detectable particle-bound binding agent-based device for detecting analytes in a multi-analyte sample utilizing two or more populations of detectable particles, wherein a first population of detectable particles are particles from about 20 nm to about 60 nm in diameter and a second population of detectable particles are particles from greater than about 60 nm to about 120 nm in diameter. In certain embodiments, the first population of detectable particles are bound to one or more binding agents having specificity for a first population of analytes and the second population of detectable particles are bound to one or more binding agents having specificity for a second population of analytes.

In a seventh aspect, the invention provides a lateral flow device for detecting bacteria in a sample, the device comprising a flow path for the sample and further comprising a binding agent that specifically binds a bacterial antigen, wherein the binding agent is immobilized on two or more populations of detectable particles. In certain embodiments, a first population of particles comprises particles from about 20 nm to about 60 nm in diameter and a second population of particles comprises particles from greater than about 60 nm to about 120 nm in diameter. In further embodiments, the first population of particles is bound to one or more binding agents having specificity for a first population of analytes and the second population of particles is bound to one or more binding agents having specificity for a second population of analytes. In still further embodiments, a capture binding agent that is immobilized on the flow path captures the one or more population of particles. In these embodiments, the population of detectable particles is disposed along the flow path such that the sample contacts the population of detectable particles before contacting the capture binding agent.

In an eighth aspect, the invention provides a method for detecting analytes in a sample, comprising contacting the sample with one or more binding agents specific for one or more analytes. In these embodiments, the one or more binding agents is immobilized on two or more population of detectable particles, wherein a first population of detectable particles comprises particles from about 20 nm to about 60 nm in diameter and a second population of detectable particles comprises particles from greater than about 60 nm to about 120 nm in diameter. In further embodiments, the first population of detectable particles is bound to a population of one or more binding agents having specificity for a first population of analytes and the second population of detectable particles is bound to a population of one or more binding agents having specificity for a second population of analytes. In still further embodiments, the sample is contacted with the one or more binding agents under conditions that permit binding between the binding agent and the analyte, wherein binding of the binding agent by the analyte indicates the presence of analyte in the sample.

In a ninth aspect, the invention provides a method for increasing specificity and/or sensitivity in a binding assay to detect binding of a plurality of analytes in a multi-analyte sample to a particle-bound binding agent, comprising contacting the sample with one or more binding agents specific for one or more analytes. In these embodiments, the one or more binding agents is immobilized on two or more populations of detectable particles, wherein a first population of detectable particles comprises particles from about 20 nm to about 60 nm in diameter and a second population of detectable particles comprises particles from greater than about 60 nm to about 120 nm in diameter. In certain embodiments, the first population of detectable particles is bound to one or more binding agents having specificity for a first population of analytes and the second population of detectable particles is bound to one or more binding agents having specificity for a second population of analytes. In further embodiments, the sample is contacted with the one or more binding agents under conditions that permit binding between the binding agent and the analyte and binding of the binding agent by the analyte indicates the presence of analyte in the sample. When the invention provides a method for increasing specificity and/or sensitivity in a binding assay by using a first population of detectable particles and a second population of detectable particles that have different diameters (as described above), the increase in sensitivity and/or specificity is in comparison to the same method but with a first population of detectable particles and a second population of detectable particles that have the same diameter.

In a tenth aspect, the invention provides a binding assay for detecting the presence of bacteria from a plurality of genera in a sample comprising the step of treating the sample with enzymes to increase the sensitivity of the binding assay.

### BRIEF DESCRIPTION OF THE DRAWINGS

FIG. 1A is a graph illustrating that the use of larger colloidal gold particles results in higher signal intensity on the capture line at various numbers of particles per reaction. FIG. 1B is a photograph from a 50% dilution series of 40 nm (“current”) gold particles in a model lateral flow system where staphylococcal protein A coated particles were captured on rabbit IgG capture lines. FIG. 1C is a set of photographs from a 50% dilution series of 80 nm (“enhanced”) gold particles in the model lateral flow system where staphylococcal protein A coated particles were captured on rabbit IgG capture lines.

FIG. 2 is a photograph taken from model lateral flow strips. These results were generated from tenfold dilutions of 8 different bacterial lysates and were derived starting from a 10⁸ stock solution, and the resulting samples were processed in lateral flow strips.

### DETAILED DESCRIPTION OF THE PREFERRED EMBODIMENTS

Unless otherwise defined herein, scientific and technical terms used in this application shall have the meaning commonly understood by those skilled in the art. The techniques and procedures described or referenced herein are well understood and commonly employed using conventional methodologies that are well known and commonly used in the art.

All publications, patents and published patent applications referred to in this application are specifically incorporated by reference herein.

Each embodiment of the invention described herein may be taken alone or in combination with one or more other embodiments of the invention.

### DEFINITIONS

Unless specified otherwise, the following definitions are provided for specific terms, which are used in the above written description.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

As used herein, a “linker” is any chemical moiety that is bound to a particle and to a binding agent, including without limitation, proteins, other biomolecules and other organic chemical compounds.

As used herein, a “multivalent binding agent” is a mixture of binding agents that specifically bind substances in a multianalyte sample, i.e., that comprise multiple specificities. One example of a multivalent binding agent is a polyclonal antibody that can bind more than one antigen of a bacterium and, thus, is multivalent.

As used herein, a “multianalyte sample” is a sample containing multiple substances having binding properties different from each other i.e., a sample that contains a plurality of different binding targets. By way of non-limiting example, a multianalyte sample may be a sample containing a plurality of different bacteria or a plurality of different proteins.

As used herein, “specifically binds” means that a pan-generic antibody recognizes and binds to a particular antigen or set of antigens (e.g., a polypeptide, carbohydrate, lipid, or glycoprotein), but does not bind non-specifically to other molecules in a sample. Likewise, an antigen bound by a pan-generic antibody that specifically binds that antigen is said to be “specifically bound” by that pan-generic antibody. Preferably, a pan-generic antibody that specifically binds a ligand forms an association with that ligand with an affinity of at least 10⁶ M⁻¹, more preferably, at least 10 M⁻¹, even more preferably, at least 10⁸ M⁻¹, and most preferably, at least 10⁹ M⁻¹ either in water, under physiological conditions, or under conditions which approximate physiological conditions with respect to ionic strength, e.g., 140 mM NaCl, and pH, e.g., 7.2.

As used herein, a “pan-generic” binding agent is a binding agent that binds more than one genus of bacteria. Pan-generic binding agents are capable of detecting more than one genus of bacteria when used in the devices and methods of the invention, for example, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more genera of bacteria. In some embodiments, the pan-generic binding agent is one or more pan-generic antibody, as described for the first aspect. In some embodiments, a pan-generic binding agent specifically binds an antigen present in more than one genus of bacteria. By way of non-limiting example, an antibody that specifically binds lipopolysaccharide on two or more genera of Gram-negative bacteria is a pan-generic binding agent. Likewise, an antibody that specifically binds lipoteichoic acid (LTA) on two or more genera of Gram-positive bacteria is a pan-generic binding agent. Such pan-generic binding agents can be polyclonal or monoclonal. In some embodiments, a pan-generic binding agent comprises antibodies with different specificities in a mixture, such that the mixture binds more than one genus of bacteria. Other non-antibody molecules may serve as pan-generic binding agents if they have the capability of binding to bacterial components (e.g. antibiotics such as polymyxin bind to lipopolysaccharides of multiple genera of Gram-negative bacteria, and vancomycin can bind to components of the cell wall of Gram-positive bacteria). These molecules, with a suitable linker, could be used as pan-generic binding agents.

As used herein, “antigen” (for example, a Gram-negative bacterial antigen or a Gram-positive bacterial antigen) is used to mean any molecule, in any structural conformation which may be specifically bound by a pan-generic binding agent. The site on the antigen which is bound by the pan-generic binding agent is called a “binding site.” An antigen may be, without limitation, a protein, a glycoprotein, a carbohydrate, or a lipid.

As used herein, “Gram-positive bacteria” means a strain, type, species, or genera of bacteria that, when exposed to the Gram stain, retains the dye and is, thus, stained blue-purple.

As used herein, “Gram-negative bacteria” means a strain, type, species, or genera of bacteria that, when exposed to the Gram stain, does not retain the dye and is not stained blue-purple. The skilled practitioner will recognize that depending on the concentration of the dye and on the length of exposure, a Gram-negative bacterium may pick up a slight amount of Gram stain and become stained light blue-purple. However, in comparison to a Gram-positive bacterium stained with the same formulation of Gram stain for the same amount of time, a Gram-negative bacterium will be much lighter blue-purple in comparison to a Gram-positive bacterium.

As used herein, “blood or blood product” includes any cell found in blood or bone marrow, as well as any product derived from the blood or bone marrow including, without limitation, whole blood, red blood cells, platelets, serum, plasma, hematopoietic stem cells, and leukocytes (including lymphocytes). The ordinarily skilled biologist will understand that without addition of anti-clotting agents such as EDTA or heparin, whole blood will clot, rendering the majority of the blood cells unusable in transfusion. Accordingly, included in the term, “blood or blood product,” is blood treated with any anti-clotting agent. In addition, during the isolation of particular blood products (e.g., platelets using platelet pheresis), non-blood components, such as physiological saline may be added to the blood. Accordingly, also included in the term, “blood or blood product,” is blood to which has been added any biologically inert substance, such as physiological saline, water, or a storage nutrient solution.

### Devices

In one aspect, the invention provides a device for detecting bacteria in a sample, the device comprising a flow path for the sample and further comprising a pan-generic antibody wherein the pan-generic antibody is specific for one or more bacterial antigens, and wherein the pan-generic antibody is immobilized via a linker on a population of particles, and a capture antibody that captures the population of particles that are bound to a bacterial antigen, wherein the capture antibody is immobilized on the flow path, and wherein the population of particles are disposed along the flow path such that the sample contacts the population of particles before contacting the capture antibody. In some embodiments, the particle is a colored particle.

In certain embodiments, the device comprises two or more pan-generic antibodies, wherein each pan-generic antibody is specific for one or more bacterial antigen. In some embodiments, each pan-generic antibody is immobilized on a separate subpopulation of particles. In some embodiments, the colored particles are from about 20 nm to 120 nm in diameter. In some embodiments, the colored particles are from about 40 nm to 80 nm in diameter. In some embodiments, the one or more populations of particles comprises a first population of particles and a second population of particles. In some embodiments, the first population of particles comprises particles from about 20 nm to about 60 nm in diameter and the second population of particles comprises particles from greater than about 60 nm to about 120 nm in diameter. In some embodiments, the first population of particles comprises particles that are about 40 nm in diameter and the second population of particles comprises particles that are about 80 nm in diameter. In some embodiments, the first population of particles has one or more binding agents having a specificity for one or more first populations of antigens and the second population of particles has one or more binding agents having a specificity for one or more second populations of antigens, wherein the first one or more population of antigens and the second one or more population of antigens are different from each other. In some embodiments, the second population of particles has a binding agent that is a monoclonal antibody. In some embodiments, the particle is a colored particle. In some embodiments, the particle is a colored gold particle.

In certain embodiments, the pan-generic antibody is immobilized on the particle via a linker. In some embodiments, the linker is protein A, protein G, or protein L. In embodiments wherein the device or method comprises two or more pan-generic antibodies, at least one of the pan-generic antibodies is immobilized on the particle via a linker. In some embodiments, the particle is a colored particle. In some embodiments the linker is protein A.

In some embodiments, the protein A is present on the detectable particles at a surface density of from about 1 to about 10 molecules of protein A per 100 nm². In some embodiments, the protein A is present on the detectable particles at a surface density of from about 2 to about 5 molecules of protein A per 100 nm².

In certain embodiments, the pan-generic antibody is a polyclonal antibody, monoclonal antibody, or a combination thereof. The pan-generic antibody may specifically bind a Gram-positive bacterial antigen or a Gram-negative bacterial antigen or a combination of Gram-positive and Gram-negative bacterial antigens. In certain embodiments, the device or method of the invention comprises at least one pan-generic antibody that specifically binds a Gram-positive bacterial antigen and at least one pan-generic antibody that specifically binds a Gram-negative bacterial antigen. In certain embodiments, the device or method of the invention comprises at least one, at least two, at least three, at least four, at least five, at least six, at least seven, at least eight, at least nine, or at least ten pan-generic antibodies that bind to a Gram-positive bacterial antigen. In certain embodiments, the device or method of the invention comprises at least one, at least two, at least three, at least four, at least five, at least six, at least seven, at least eight, at least nine, or at least ten pan-generic antibodies that bind to a Gram-negative bacterial antigen. In certain embodiments, the device or method of the invention comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty pan-generic antibodies, wherein the pan-generic antibodies are a mix of pan-generic antibodies that bind to a Gram-positive bacterial antigen and pan-generic antibodies that bind to a Gram-negative bacterial antigen. In some embodiments, antibodies that bind a Gram-negative bacterial antigen are immobilized on a separate subpopulation of particles than antibodies that bind Gram-positive bacterial antigens so that the presence or absence of Gram-negative and Gram-positive bacteria can be detected separately.

A pan-generic binding agent may comprise one or more polyclonal antibodies wherein the polyclonal antibodies are directed against one antigen or multiple antigens. A pan-generic binding agent may comprise one or more monoclonal antibodies or a combination of polyclonal and monoclonal antibodies. In some embodiments, a polyclonal antibody and monoclonal antibodies are immobilized on separate subpopulations of particles. In embodiments comprising a plurality of monoclonal antibodies with different specificities, each specificity may be immobilized on a separate subpopulation of particles. In embodiments comprising multiple polyclonal antibodies with different specificities, each specificity may be immobilized on a separate subpopulation of particles. In some embodiments, the subpopulations of particles are different sizes, colors or both.

In some embodiments, a capture binding agent is a polyclonal antibody, monoclonal antibody, or a combination thereof. In certain embodiments, a capture antibody is a pan-generic antibody that specifically binds a bacterial antigen bound by the pan-generic antibody immobilized on a particle. In certain embodiments, a capture antibody is the same as a pan-generic antibody immobilized on a particle.

In some embodiments, the invention provides a device that is a lateral flow device. In some embodiments, the invention provides a device comprising one or more absorbent membranes. Those of skill in the art will be familiar with materials suitable for use as an absorbent membrane in such devices. In certain embodiments, the absorbent membrane is a nitrocellulose membrane. In some embodiments, the invention provides a device comprising a flow path on which one or more capture antibodies are immobilized. In certain embodiments, two or more, three or more, four or more, five or more, six or more, seven or more, eight or more, nine or more, or ten or more capture antibodies are immobilized on the flow path. In certain embodiments, the capture antibodies are immobilized in one or more locations on the flow path. In specific embodiments, the capture antibodies are immobilized in two or more, three or more, four or more, five or more, six or more, seven or more, eight or more, nine or more, or ten or more locations on the flow path. In some embodiments, each of the one or more locations comprises the same capture antibody. In some embodiments, each of the one or more locations comprises different capture antibodies.

In some embodiments, the invention provides a device comprising pan-generic antibody that is immobilized via a linker on a population of detectable particles, wherein the particles are dried within a support surface disposed above an absorbent membrane and in contact with the upper surface of the membrane where the area of contact between the support surface and the absorbent membrane controls the rate of reconstitution of the particles and/or the time between reconstitution and contacting a capture antibody. In some embodiments, the detectable particle is a chemiluminescent, a luminescent, a fluorescent, a magnetic or a colored particle. In embodiments utilizing a colored particle, the particle may be a gold, silver, or platinum particle. In some embodiments, the particle is from about 20 to about 120 nm in diameter. In some embodiments the particle is from about 40 to about 80 nm in diameter.

In some embodiments, the device comprises a positive control. In some embodiments, the device comprises a location on a flow path indicating that the sample has flowed past the capture antibodies.

In certain embodiments, such a pan-generic binding agent comprises an antibody which binds under physiological conditions to an antigen-containing epitope of a lipopolysaccharide (LPS) structure of a Gram-negative bacteria or a lipoteichoic acid (LTA) structure of a Gram-positive bacteria.

Pan-generic antibodies useful in the devices and methods of the invention include a monoclonal antibody, a polyclonal antibody, a single-chain antibody, a synthetic antibody, a recombinant antibody, a chimeric antibody, or any antigen-binding fragment of the above, including, but not limited to, F(ab), F(ab′), F(ab′)₂, scFv fragments and recombinant fragments. The skilled worker will understand that these fragments are to be functional fragments when used in the devices and methods of the invention. The pan-generic antibodies may be from non-mammalian species, for example, a chicken antibody, or from a mammalian species, including but not limited to rabbits, rodents (including mice, rats and guinea pigs), goats, pigs, sheep, camelids and humans. The pan-generic antibodies also may be humanized or chimeric antibodies.

Those skilled in the art are enabled to make any such antibody derivatives using standard art-recognized techniques. For example, Jones et al. (Nature 321: 522-525 (1986)) discloses replacing the CDRs of a human antibody with those from a mouse antibody. Marx (Science 229: 455-456 (1985)) discusses chimeric antibodies having mouse variable regions and human constant regions. Rodwell (Nature 342: 99-100 (1989)) discusses lower molecular weight recognition elements derived from antibody CDR information. Clackson (Br. J. Rheumatol. 3052: 36-39 (1991)) discusses genetically engineered monoclonal antibodies, including Fv fragment derivatives, single chain antibodies, fusion proteins chimeric antibodies and humanized rodent antibodies. Reichman et al. (Nature 332: 323-327 (1988)) discloses a human antibody on which rat hypervariable regions have been grafted. Verhocyen et al. (Science 239: 1534-1536 (1988)) teaches grafting of a mouse antigen binding site onto a human antibody.

Most preferably, the pan-generic antibodies of the present invention are polyclonal antibodies or monoclonal antibodies. Generation of monoclonal and polyclonal antibodies is well within the knowledge of one of ordinary skill in the art of biology (see, e.g., Ausubel et al., Current Protocols in Molecular Biology, John Wiley & Sons, New York, N. Y., 1994). A number of procedures are useful in producing antibodies to the desired unique target antigens. Traditional immunization and harvesting techniques will result in the creation of polyclonal antibodies directed against the common determinants of the target bacterial species including pan-generic determinants such as LPS and LTA. Additionally, cellular hybridization techniques can be utilized to produce immortal hybridoma cell lines that generate specific monoclonal antibodies to the target species.

Antibodies having potential utility for broadly detecting Gram-positive bacteria include those described in Fisher et al., PCT Publication No. WO98/57994; Jackson, D. E. et al., Infection and Immunity 43: 800 (1984); Hamada. S. et al, Microbiol. Immunol. 28: 1009 (1984); Aasjord, P. et al., Acta Path. Microbiol. Immunol. Scand. Sect. C, 93: 245 (1985); McDaniel, L. S. et al., Microbial Pathogenesis 3: 249 (1987); Tadler, M. B. et al., Journal of Clinical Laboratory Analysis 3: 21 (1989); and Stuertz, K et al., Journal of Clinical Microbiology 36: 2346 (1998).

Antibodies having potential utility for broadly detecting Gram-negative bacteria include those described in Nelles, M. J. et al, Infect. Immun. 46: 677 (1984); Teng. N. N. H. et al, Proc. Natl. Acad. Sci. USA 82: 1790 (1985); Dunn, D. L. et al., Surgery 98: 283 (1985); De Jongh-Leuvenink, J. et al, Eur. J. Clin. Microbiol. 5: 148 (1986); Bogard, W. C. et al., Infect. Immun. 55: 899 (1987); Pollack, M. et al., Bacterial Endotoxins: Pathophysiological Effects, Clinical Significance, and Pharmacological Control. pp. 327-338 Alan R. Liss, Inc. (1988); Priest, B. P. et al., Surgery 106: 147 (1989); Tyler, J. W. et al., Journal of Immunological Methods 129: 221 (1990); Siegel, S. A. et al., Infect. Immun. 61: 512 (1993); Shelburne, C. E. et al., J. Periodont. Res. 28: 1 (1993); Di Pardova. F. E. et al., Infect. Immun. 61: 3863 (1993); and De Kievit, T. R. and Lam, J. S. J. Bacteriol. 176: 7129 (1994).

The selection as to which antibody or antibodies to use can be accomplished through classical techniques. Antibody specificity, binding extent and kinetics can be characterized by empirically testing each antibody in an empirical format. Micro-titer screening formats are well documented in the literature to aid in characterizing specific antibody response in any given immunoassay format. Likewise, the activities of detectably labeled antibodies can be characterized by executing a variety of chemical conjugation techniques and screening the resulting product for the optimal performance parameters. The capture antibody and detectably labeled antibody can be screened against the clinical isolates of bacteria from retained platelet or red cell samples to emulate final assay performance as close to final product embodiment as possible. This experimentation leads to the selection and optimization of antibody reagents for application in the various assay formats described below.

Monoclonal antibodies with specificity towards cross-genus targets on the bacterial cell surfaces may be utilized in devices and methods of the invention. In some embodiments, blends of monoclonal antibodies may be utilized. Polyclonal antibodies, including polyclonal antisera or polyclonal mixtures made by blending monoclonal and/or polyclonal antibodies with broad specificity across the different Gram-negative and Gram-positive species are useful in the devices and methods of the invention.

The antibodies indicated above can be utilized as described or modified as necessary to produce a useful immunological reagent.

In some embodiments, the particles useful in the binding assays and lateral flow device of the invention are one or more of gold, silver, or platinum particles. The particles can be of a uniform size, or they can be multiple sizes. In some embodiments, the particles can have a size of 10 nm to 150 nm, for example from 20 nm to 50 nm, from 40 nm to 80 nm, or from 60 nm to 100 nm. Exemplary particle sizes include 10 nm, 20 nm, 30 nm, 40 nm, 50 nm, 60 nm, 70 nm, 80 nm, 90 nm, 100 nm, 110 nm, 120 nm, 130 nm, 140 nm, and 150 nm. In certain embodiments, at least some of the particles are sized from about 60 nm to about 120 nm. In certain embodiments, all of the particles are sized from about 20 nm to about 120 nm. In certain embodiments, the particle size is from about 40 to about 80 nm. In other embodiments, the particle size is about 40 nm. In other embodiments, the particle size is about 40 nm. In yet other embodiments, the device or method may comprise subpopulations of particles having different sizes, e.g., a subpopulation of 40 nm particles and a subpopulation of 80 nm particles. In certain embodiments, the device or method may comprise 40 nm particles and 80 nm particles, wherein a monoclonal antibody (e.g., a pan-generic monoclonal antibody) is immobilized to the 40 nm particles and a polyclonal antibody (e.g., a pan-generic polyclonal antibody) is immobilized to the 80 nm particles. Different bead sizes can be used to create different signals, for example, to distinguish between binding to different targets. Without wishing to be bound by theory, smaller particle sizes may be better used with monoclonal antibodies because a larger quantity of smaller beads will produce more surface area than a smaller quantity of larger beads. For the monoclonal antibodies, efficient capture will be much more important than avidity. By contrast, avidity becomes much more important with larger beads, as discussed below.

In certain embodiments, the linker between a particle and a pan-generic binding agent is a protein linker (e.g., Protein L, Protein A, Protein G, or Protein A/G), or biotin-avidin, streptavidin, or neutravidin, or a species-specific anti-immunoglobulin antibody to immobilize another antibody on the conjugate (e.g., an anti-rabbit-immunoglobulin antibody or an anti-mouse-immunoglobulin antibody), agents capable of binding a recombinant protein tag (e.g., a His tag or a FLAG tag), DNA or a DNA-like molecule, or a synthetic immunoglobulin-binding moiety (e.g., a ProMetric BioSciences mimetic ligand). In particular embodiments, the linker is protein A. Without wishing to be bound by theory, a single antibody can be bound to two linkers, thereby providing a basis for the avidity cascade discussed below. The skilled worker also will appreciate that the linkers of the invention are useful in a variety of assays using solid supports, for example, arrays, ELISAs, cantilever-based systems, SPR, and Luminex® assays.

The present invention provides a surprising use for linkers used to bind binding agents such as antibodies. Antibody-binding linkers are disfavored in serum- or plasma-based assays because these linkers can interact with native antibodies in the sample, interfering with the antibodies used in the assay itself. Here, though, the off rate of the binding agents of the invention to the linkers is insignificant compared to the time frame to run the assay (typically 20-30 minutes). Thus, the present invention is able to utilize linkers such as protein A without concern over the competition from native human antibodies present in a sample.

In some embodiments, the device is a lateral flow device suitable for use in detecting bacteria in a blood sample or a blood product sample, the device comprising a flow path for the sample and a pan-generic binding agent (e.g., a pan-generic antibody or functional fragment thereof as described herein) that binds a plurality of bacterial antigens, wherein the pan-generic binding agent is immobilized via a linker on a population of 80 nm gold particles, and further comprising a pan-generic binding agent (e.g., a pan-generic antibody or functional fragment thereof as described herein) that is immobilized via a linker on a population of 40 nm gold particles. In further embodiments, the pan generic binding agents bind one or more Gram-positive bacterial antigens, one or more Gram-negative bacterial antigens, or both. In various embodiments, a pan-generic binding agent that binds a Gram-positive bacterial antigen may be on the same population or on a different population of gold particles (e.g., 80 nm gold particles) as a pan-generic binding agent that binds a Gram-negative bacterial antigen. In certain embodiments, a pan-generic binding agent immobilized on an 40 nm gold particle is a monoclonal antibody and a pan-generic binding agent immobilized on an 80 nm gold particle is a polyclonal antibody. In further embodiments, the device comprises a capture binding agent (e.g., a capture antibody) immobilized on the flow path of the device, wherein the gold particles are disposed along the flow path such that the sample contacts the population of colored particles before contacting the capture binding agent. In certain embodiments, the capture binding agent is a pan-generic binding agent. In embodiments in which the capture binding agent is a pan-generic binding agent, the capture binding agent may be the same as the pan-generic binding agent immobilized on the gold particles or may be different from the pan-generic binding agent immobilized on the gold particles.

The device according to the invention provides greater sensitivity than prior art devices. Without wishing to be bound by theory, the device according to the invention is believed to provide greater sensitivity because the linker (e.g., protein A) initiates an avidity cascade. Multivalent antigens can be bound by more than one particle, thereby bringing particles in proximity to each other. This creates a localized higher concentration of binding agents, which in turn causes more antigen to be bound and more particles brought into proximity with each other. Eventually, this particle to particle avidity causes numerous particles to be brought into proximity, creating an agglomeration of particles that is highly detectable. This aggregation can be aided by the natural equilibrium of antibody-linker binding. Antibodies will naturally bind to and be released from the linker. This phenomenon results in a fraction of particle-bound linkers that is free of antibody. These antibody-free linkers can bind antibodies such as those already bound to linkers on other beads, thereby triggering the avidity cascade.

In a fifth aspect, the invention provides a method for making a particle that has a very high surface density of linker, and thus a very high surface density of binding agent. The method according to this aspect comprises incubating detectable particles with a high concentration of linker. For example, gold, silver, or titanium particles can be incubated with a solution of protein A, wherein the solution has a concentration of at least about 0.1 μg of protein A and can be as high as a saturated solution of protein A. The detectable particles have an optical density (OD) that is characteristic to the particular particle material, which can be used to determine the concentration of particles in solution. In some embodiments, the method according to this aspect of the invention utilizes a solution has a concentration of protein A from about 0.1 μg/mL to about 0.4 μg per OD unit of particle.

In some embodiments, the invention provides a detectable particle-bound binding agent-based device for detecting analytes (e.g., bacterial antigens) in a multi-analyte sample. In certain embodiments, the device utilizes two or more populations of detectable particles of different sizes. For example, a first population of detectable particles can be particles from about 20 nm to about 60 nm in diameter (e.g., about 40 nm in diameter) and a second population of detectable particles can be particles from greater than about 60 nm to about 120 nm in diameter (e.g., about 80 nm in diameter). In particular embodiments, the first population of detectable particles is bound to one or more binding agents having specificity for a first population of analytes and the second population of detectable particles are bound to one or more binding agents having specificity for a second population of analytes. The first population and the second population of analytes can be the same analytes or they can be different analytes. When the populations of analytes are different, the analytes of the first population of analytes can be present in the sample at a higher concentration than analytes of the second population of analytes. Without wishing to be bound by theory, applicants hypothesize that using larger particles when trying to detect rare analytes in a sample increases sensitivity by triggering an avidity cascade whereby the particles bound to the analyte cooperatively recruit other particles, resulting in a larger detection signal.

In some embodiments, the invention provides a lateral flow device for detecting bacteria in a sample. In certain embodiments, the device comprises a flow path for the sample and a binding agent (e.g., an antibody or functional fragment thereof as described herein) that specifically binds a bacterial antigen. In certain embodiments, the binding agent is immobilized on two or more populations of detectable particles. In these embodiments, a first population of particles can comprise particles from about 20 nm to about 60 nm in diameter (e.g., about 40 nm in diameter) and a second population of particles can comprise particles from greater than about 60 nm to about 120 nm in diameter (e.g., about 80 nm in diameter). In particular, the first population of particles can be bound to one or more binding agents having specificity for a first population of analytes and the second population of particles can be bound to one or more binding agents having specificity for a second population of analytes. The first population and the second population of analytes can be the same analytes or they can be different analytes. When the populations of analytes are different, the analytes of the first population of analytes can be present in the sample at a higher concentration than analytes of the second population of analytes. Without wishing to be bound by theory, applicants hypothesize that using larger particles when trying to detect rare analytes in a sample increases sensitivity by triggering an avidity cascade whereby the particles bound to the analyte cooperatively recruit other particles, resulting in a larger detection signal. In certain embodiments, a capture binding agent is capable of capturing the one or more population of particles, wherein the capture binding agent is immobilized on the flow path of the lateral flow device and the population of detectable particles is disposed along the flow path such that the sample contacts the population of detectable particles before contacting the capture binding agent.

In certain embodiments, the detectable particles (first population or second population) are made of a material selected from gold, silver and platinum. In particular embodiments, the detectable particles are gold particles.

In certain embodiments, the binding agents are bound to the detectable particles via a linker, e.g., protein A, protein G, or protein L. In these embodiments, the linker may be present on the detectable particles at a surface density of from about 1 to about 10 molecules of linker per 100 nm².

In certain embodiments, the one or more binding agents bound to the first population of detectable particles comprises one or more antibodies (e.g., monoclonal antibodies), or functional fragments thereof as described herein. In certain embodiments, the one or more binding agents bound to the second population of detectable particles comprises one or more antibodies (e.g., polyclonal antibodies), or functional fragments thereof as described herein. The skilled worker will appreciate that binding agents can be selected from polyclonal antibodies, monoclonal antibodies, functional fragments thereof, and combinations thereof, and that the antibodies may comprise one or more pan-generic antibodies.

### Methods of Detecting Bacteria

In some embodiments, the invention provides a device and method with broader reactivity than existing devices and methods. In particular, the devices and methods are capable of detecting a broader range of bacterial genera, species, and/or strains of bacteria than existing devices and methods. For example, the devices and methods may be capable of detecting at least 100, 150, 200, 250, 300, 350, 400, 450, or 500 different bacteria. In some embodiments, the invention provides a method or device comprising a pan-generic antibody capable of detecting greater than 1×10⁷, 1×10⁶, 1×10⁵, 1×10⁴, 1×10³, or 1×10² colony forming units (CFU) per mL of bacteria or an equivalent concentration of antigens derived from that level of bacteria.

In some embodiments, the invention provides a method to screen for the presence of bacteria in a liquid sample. In various embodiments of the method, the sample may be any biological fluid, including a dialysis sample. In some embodiments, the dialysis sample is selected from hemodialysis fluid and peritoneal dialysis fluid. In some embodiments, the sample is a sample of fluid in which a tissue has been stored. In some embodiments, the tissue is selected from the group consisting of blood cell cultures, stem cell cultures, and bone and cartilage graft materials. In some embodiments the sample is blood or a blood product including but not limited to whole blood, leukocytes, hematopoietic stem cells, platelets, red blood cells, plasma, bone marrow and dialysis fluid, comprising contacting a lateral flow device of the invention with a sample and detecting binding of the populations of antibodies to the sample, wherein binding indicates the presence of bacteria in the sample and no binding indicates the absence of bacteria in the sample. In certain embodiments, the sample is a dialysis fluid including hemodialysis fluid or peritoneal dialysis fluid.

In some embodiments, the invention provides a method to screen for the presence of bacteria in food or beverage products or food or beverage processing. For example, the methods of the invention could be used to test for the presence or absence of bacteria in lines used to carry liquids such beer or milk. The methods also could be used to test for the presence or absence of bacteria in water samples. In some embodiments, these methods comprise contacting a lateral flow device of the invention with a sample of a beverage or water sample and detecting binding of the populations of antibodies to the sample, wherein binding indicates the presence of bacteria in the beverage or water sample and no binding indicates the absence of bacteria in the beverage or water sample.

In certain embodiments of the invention, the sample is treated prior to or concomitantly with contacting the sample with a pan-generic antibody. Preferably, the treatment exposes a binding site for the pan-generic antibody on the Gram-negative bacterial antigen or on the Gram-positive bacterial antigen. A binding site on a bacterial antigen may be exposed by, for example, cleaving an antigen from the cell wall or cell membrane of the bacteria, thereby exposing the binding site; inducing the bacteria to secrete the antigen, thereby exposing the binding site; lysing the bacteria, thereby releasing an intracellular bacterial antigen and thus exposing the binding site on the antigen; or by inducing a conformational change on the bacterial antigen, thereby exposing the binding site. Such treatments include mechanical disruption of the bacterial cells in the sample by physical means, including, without limitation, sonication, boiling, or homogenization using, for example, a Dounce homogenizer. The treatment may also be treatment of the sample by chemical means with a compound or composition, such as detergent, a basic solution (for alkaline lysis), an acidic solution (for acidic lysis), EDTA, EGTA, a metal ion, an anion, a cation, a surfactant, a chelator, and/or an enzyme (e.g., lysostaphin, lysozyme, mutanolysin, labiase, achromopeptidase, trypsin, proteinase K, an autolysin, bacteriophage-encoded lytic enzymes, and combinations thereof). The treatment exposes a binding site for the pan-generic antibody on the Gram-negative bacterial antigen or on the Gram-positive bacterial antigen.

In a further embodiment, the sample is contacted with enzymes to increase the sensitivity or efficiency of the binding assay. In various embodiments, the sample can be any liquid sample that is suspected of containing bacteria. In some embodiments, the sample is a biological fluid, including urine, sputum, spinal fluid, ascites, blood, or blood products. In some embodiments, the sample may be lavage fluid, or products, including blood products for administration to a subject in need thereof. In some embodiments, the sample is blood or a blood product. In some embodiments the blood or blood product is selected from the group consisting of: whole blood, leukocytes, hematopoietic stem cells, platelets, red blood cells, plasma, bone marrow and serum.

According to this embodiment, the sample is treated with one or more enzymes that increase the amount, availability, and/or dispersal of targets for the binding reagents described herein in a binding assay. In various embodiments, the treatment can be prior to contacting the sample with binding reagents (i.e., pretreating the sample) or the sample may be contacted with the enzymes and the binding reagents at the same time.

According to this embodiment, binding reagents may be, without limitation, an antibody or a functional fragment thereof as described herein, an antibiotic, a protein, a fusion protein (i.e., a protein comprising portions of two or more proteins), or a chemical chelator. In certain embodiments, a binding agent according to the invention is a peptide, a protein, or a peptidomimetic. In some embodiments, the binding reagent is a peptidoglycan binding protein or a β-glucan-binding protein, such as those found in silkworm larvae plasma. See, e.g., U.S. Pat. No. 7,598,054 the contents of which are incorporated herein by reference in its entirety for all purposes. In a particular embodiment, the assay is an immunoassay, i.e., the binding reagents are antibodies or functional fragments thereof.

In some embodiments, the sample is treated with one or more enzymes selected from the group consisting of lysostaphin, lysozyme, mutanolysin, labiase, lipase, achromopeptidase, trypsin, proteinase K, an autolysin, bacteriophage-encoded lytic enzymes, and combinations thereof.

In some embodiments, the enzymes are specific for peptidoglycans. In some embodiments, the one or more enzymes comprise a bacterial cell wall-specific endopeptidase, a bacterial cell wall-specific glycoside hydrolase, or combinations thereof.

In some embodiments, the one or more enzymes is a bacterial cell wall-specific endopeptidase. By way of illustration, the bacterial cell wall-specific endopeptidase can include but is not limited to lysostaphin, and achromopeptidase. In some embodiments, the bacterial cell wall-specific endopeptidase is present in a concentration of from about 200 to about 0.06 units/ml. In some embodiments, treatment is with one or more enzymes including lysostaphin. In some embodiments, the lysostaphin is present in a concentration of from about 200 to about 0.06 units/ml.

In some embodiments, the one or more enzymes is a bacterial cell wall-specific glycoside hydrolase. By way of illustration, bacterial cell wall-specific glycoside hydrolase can include but is not limited to lysozyme and mutanolysin. In some embodiments, the bacterial cell wall-specific glycoside hydrolase is present in a concentration of from about 15 to about 0.0025 mg/ml. In some embodiments, treatment is with one or more enzymes including lysozyme. In some embodiments, the lysozyme is present in a concentration of from about 15 to about 0.0025 mg/ml.

In some embodiments, the enzymes used for treatment of the sample comprise a bacterial cell wall-specific endopeptidase and a bacterial cell wall-specific glycoside hydrolase. In some embodiments, the treatment is with lysostaphin and lysozyme.

The inventors have surprisingly found that treatment of a sample with lysostaphin and lysozyme increases the sensitivity of a bacterial detection immunoassay. The addition of lysostaphin and lysozyme can increase the sensitivity of Gram positive and Gram negative bacterial detection by approximately 10-fold to 100-fold.

In some embodiments, the sample is treated with a combination of agents that comprises enzymes and one or more surfactants.

In some embodiments, the sample is incubated with the one or more enzymes (with or without a surfactant) prior to the step of detecting binding. In particular embodiments, the incubation step is eliminated so that detection of the presence or absence of binding is assessed directly after contacting the sample with the enzymes and the binding reagents.

In some embodiments, the method is for use in detecting bacteria in a blood sample or a blood product sample, the method comprising contacting the sample with a pan-generic binding agent (e.g., an antibody or functional fragment thereof as described herein, such as a pan-generic antibody) that binds a plurality of bacterial antigens, wherein the pan-generic binding agent is immobilized via a linker on a population of larger (e.g., 80 nm) gold particles, and further comprising a pan-generic binding agent (e.g., a pan-generic antibody) that is immobilized via a linker on a population of smaller (e.g., 40 nm) gold particles. In some embodiments, the larger particles have one or more monoclonal binding agent immobilized on them via a linker. In various embodiments, the method comprises contacting the sample with the pan-generic binding agent under conditions that permit binding between the pan-generic binding agent and the bacterial antigen and contacting an immobilized capture binding agent (e.g., a pan-generic binding agent such as a pan-generic antibody) with the gold particle under conditions that permit binding between the immobilized capture binding agent and the gold particle with the immobilized pan-generic binding agent. In certain embodiments, pan-generic binding agents bind one or more Gram-positive bacterial antigens, one or more Gram-negative bacterial antigens, or both. In various embodiments, a pan-generic binding agent that binds a Gram-positive bacterial antigen may be on the same population or on a different population of gold particles (e.g., 80 nm gold particles) as a pan-generic binding agent that binds a Gram-negative bacterial antigen. In certain embodiments, a pan-generic binding agent immobilized on an 80 nm gold particle is a polyclonal antibody and a pan-generic binding agent immobilized on a 40 nm gold particle is a monoclonal antibody. In certain embodiments, a pan-generic binding agent immobilized on an 80 nm gold particle is a monoclonal antibody and a pan-generic binding agent immobilized on a 40 nm gold particle is a polyclonal antibody. In further embodiments in which the capture binding agent is a pan-generic binding agent, the capture binding agent is the same as the pan-generic binding agent immobilized on the gold particles or is different from the pan-generic binding agent immobilized on the gold particles.

In a further aspect, the invention provides a kit comprising a detectable particle, such as a colored particle, including a gold, silver or platinum particle wherein the particle is sized about 20 nm to about 120 nm and wherein the particle comprises a multivalent binding agent immobilized thereon via a linker. In some embodiments, the multivalent binding agent is pan-generic binding agent such as a pan-generic antibody for the detection of Gram-negative bacteria, Gram-positive bacteria or both in a sample. In some embodiments, the particle is about 80 nm. In some embodiments, the kit comprises detectable particles of different sizes, such as 80 nm and 40 nm. In some embodiments, the kit comprises 80 nm gold particles with or without 40 nm gold particles. The kit further comprises instructions for using the detectable particle to detect the presence of bacteria in a sample. In some embodiments, the kit further comprises a solid surface having a capture pan-generic antibody immobilized thereon. In some embodiments, the solid surface is a component of a lateral flow device. In some embodiments, the kit further comprises a reagent for pretreating a sample.

In some embodiments, the invention provides a method for detecting analytes (e.g., bacterial antigens) in a sample by contacting the sample with one or more binding agents (e.g., an antibody or functional fragment thereof as described herein, including pan-generic binding agents) specific for one or more analytes. In certain embodiments, the one or more binding agents is immobilized on two or more population of detectable particles. In these embodiments, a first population of detectable particles comprises particles from about 20 nm to about 60 nm in diameter (e.g., 40 nm in diameter) and a second population of detectable particles comprises particles greater than about 60 nm to about 120 nm in diameter (e.g., 80 nm in diameter). In certain embodiments, the first population of detectable particles is bound to a population of one or more binding agents having specificity for a first population of analytes and the second population of detectable particles is bound to a population of one or more binding agents having specificity for a second population of analytes. The method also comprises contacting the sample with the one or more binding agents under conditions that permit binding between the binding agent and the analyte, wherein binding of the binding agent by the analyte indicates the presence of analyte in the sample. In certain embodiments, the method further comprises contacting an immobilized capture binding agent with the detectable particle under conditions that permit binding between the immobilized capture binding agent and the detectable particle with the binding agent. In these embodiments, binding of the detectable particle by the capture binding agent indicates the presence of analyte in the sample.

In some embodiments, the invention provides a method for increasing specificity and/or sensitivity in a binding assay to detect binding of a plurality of analytes (e.g., bacterial antigens) in a multi-analyte sample to a particle-bound binding agent. In these embodiments, the method comprises contacting the sample with one or more binding agents (e.g., pan-generic binding agents) specific for one or more analytes, wherein the one or more binding agents is immobilized on two or more populations of detectable particles. In certain embodiments, a first population of detectable particles comprises particles from about 20 nm to about 60 nm in diameter (e.g., 40 nm in diameter) and a second population of detectable particles comprises particles greater than about 60 nm to about 120 nm in diameter (e.g., 80 nm in diameter). Because the first population of particles is from about 20 nm to about 60 nm in diameter and the second population of detectable particles is greater than about 60 nm to about 120 nm in diameter, the first population of detectable particles and the second population of detectable particles do not have the same diameter. In these embodiments, the first population of detectable particles is bound to one or more binding agents having specificity for a first population of analytes and the second population of detectable particles is bound to one or more binding agents having specificity for a second population of analytes. When the sample is contacted with the one or more binding agents under conditions that permit binding between the binding agent and the analyte, binding of the binding agent by the analyte indicates the presence of analyte in the sample. Detection of this binding will inform the skilled worker of the presence of analyte in the sample. The increase in sensitivity and/or specificity is in comparison to this same method but with a first population of detectable particles and a second population of detectable particles that have the same diameter.

In certain embodiments, the first population and the second population of analytes can be the same analytes or they can be different analytes. When the populations of analytes are different, the analytes of the first population of analytes can be present in the sample at a higher concentration than analytes of the second population of analytes. Without wishing to be bound by theory, applicants hypothesize that using larger particles when trying to detect rare analytes in a sample increases sensitivity by triggering an avidity cascade whereby the particles bound to the analyte cooperatively recruit other particles, resulting in a larger detection signal.

In certain embodiments, the detectable particles (first population or second population) are made of a material selected from gold, silver and platinum. In particular embodiments, the detectable particles are gold particles.

In certain embodiments, the binding agents are bound to the detectable particles via a linker, e.g., protein A, protein G, protein L, or a species-specific anti-immunoglobulin antibody. In these embodiments, the linker may be present on the detectable particles at a surface density of from about 1 to about 10 molecules of linker per 100 nm².

In certain embodiments, the one or more binding agents bound to the first population of detectable particles comprises one or more antibodies (e.g., monoclonal antibodies). In certain embodiments, the one or more binding agents bound to the second population of detectable particles comprises one or more antibodies (e.g., polyclonal antibodies). The skilled worker will appreciate that binding agents can be selected from polyclonal antibodies, monoclonal antibodies, functional fragments thereof, and combinations thereof, and that the antibodies may comprise one or more pan-generic antibodies.

The skilled worker will appreciate that any of the embodiments described herein can be combined with any other embodiment or combination of embodiments also described herein.

The following examples are intended to further illustrate certain embodiments of the invention and are not intended to limit the scope of the invention.

### EXAMPLES

### Example 1

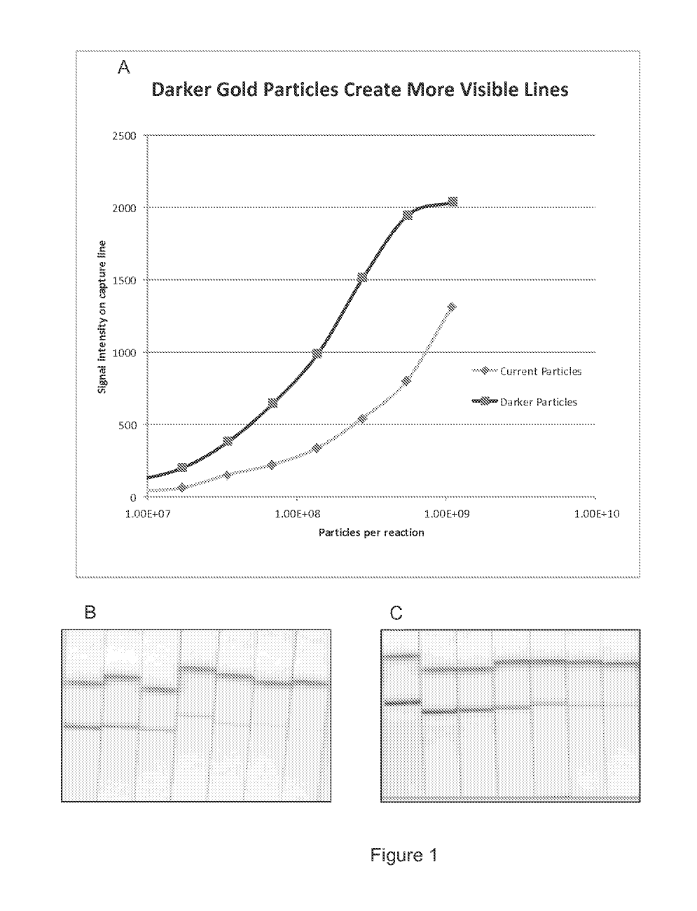

We measured the visual signal generated from different sized (40 nm and 80 nm) gold particles in a model lateral flow system to determine which particles gave the greatest signal intensity response per particle (FIGS. 1A-1C). The system was designed to capture a high proportion of the particles flowing through the strip, to give an indication of the visual signal produced by particles of varying sizes. A lateral flow device according to the invention was used. In this model the flow device utilized an IgG antibody striped on a Millipore nitrocellulose membrane as a capture binding agent, and protein A coated gold particles flowing through the strip. For a given number of particles added to the reaction. An 80 nm gold particle resulted in a higher contrast intensity purple line as compared to the red/pink line produced by the 40 nm gold particles, making the lines from the 80 nm beads easier to visualize and interpret. FIGS. 1B and 1C are images of the strips produced using varying numbers of 40 nm and 80 nm particles, respectively. The images were analyzed using Gelanalyzer 2010 software to provide values for the intensity of the capture lines. FIG. 1A shows a plot of signal intensity vs. the number of particles added to the reactions, demonstrating the increased signal intensity produced by equal numbers of larger gold particles.

Surprisingly, increasing the size of the particle also increases the intensity of the visually detectable signal generated on the capture line, thereby increasing the sensitivity. However, more numerous smaller particles, which have a larger surface to volume ratio than larger particles, would be expected to yield a better signal with faster results. Additionally, the amount of gold in the capture area is limiting, as a practical matter. Thus, it was particularly surprising that a lower amount of larger particles yielded better results than a higher amount of smaller particles.

### Example 2

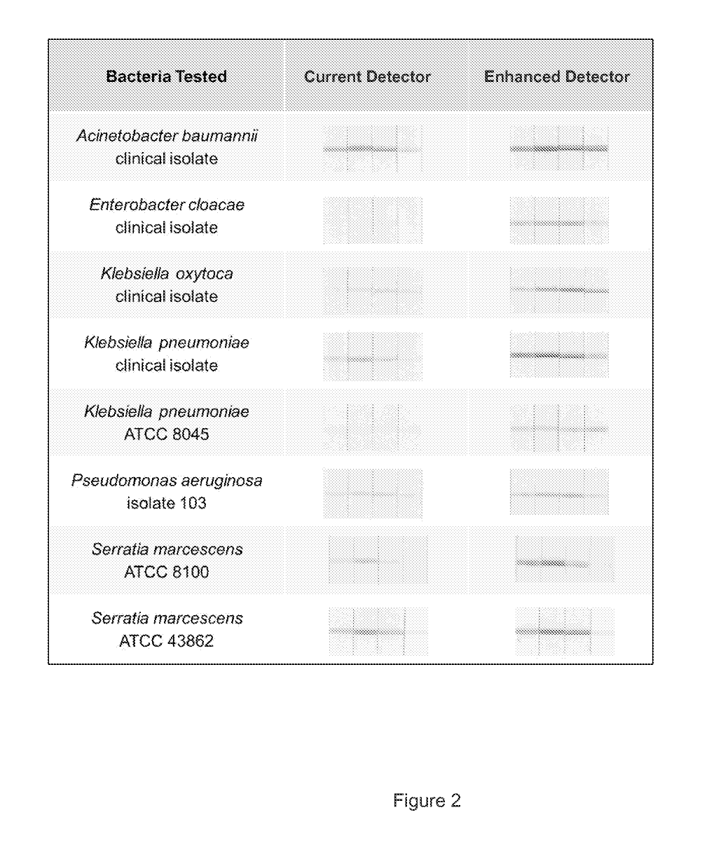

To test the effectiveness of the gold particles of different sizes, we constructed a model immunoassay system using a mixture of antibodies raised against a variety of Gram-negative and Gram-positive bacteria. We coupled the antibodies to 80 nm colloidal gold (“enhanced detector”) particles and compared their performance to 40 nm colloidal gold (“current detector”) particles. We prepared four levels of bacterial lysates for each of eight organisms by making tenfold dilutions starting at 10⁸ CFU/mL using a buffered solution. For each lysate level, we mixed 20 μL of current detector particles or enhanced detector particles (ODS), 20 μL of bacterial lysate, and 20 μL of a running buffer containing detergents in wells of a 96-well plate. A 0.5 cm dipstick cut from a Millipore nitrocellulose membrane card striped with the same antibody and laminated to an upper absorbent wick was inserted into each well and incubated until all of the liquid flowed into the dipstick. A chase of 100 μL PBS was used to clear the dipstick so it could be visually graded for signal intensity on a 1-12 scale vs. an intensity standard (deposited dilutions of particles) (Table 1 and FIG. 2).

TABLE 1
Signal intensity of current detector particles vs. enhanced
detector particles

| | Current Polyclonal | Enhanced Polyclonal |
|---|---|---|
| Bacteria Tested | Current Detector | Enhanced Detector |
| Acinetobacter baumannii | 10, 11, 10, 4 | 10, 11, 11, 10 |
| clinical isolate | | |
| Enterobacter cloacae | 2, 2, 3, 2 | 7, 8, 9, 5 |
| clinical isolate | | |
| Klebsiella oxytoca | 3, 4, 6, 4 | 5, 8, 10, 8 |
| clinical isolate | | |
| Klebsiella pneumoniae | 5, 8, 6, 2 | 10, 11, 11, 6 |
| clinical isolate | | |
| Klebsiella pneumoniae | 3, 3, 3, 3 | 5, 6, 6, 7 |
| ATCC 8045 | | |
| Pseudomonas aeruginosa | 6, 6, 7, 3 | 4, 8, 8, 3 |
| isolate 103 | | |
| Serratia marcescens | 3, 8, 2, 0 | 9, 10, 7, 1 |
| ATCC 8100 | | |
| Serratia marcescens | 9, 10, 9, 4 | 9, 11, 10, 4 |
| ATCC 43862 | | |

In all cases, the enhanced detectors were at least as sensitive as the current detectors, and in many cases, the signal was dramatically increased with the enhanced detector particles as compared to the current detector particles. For some bacterial species we observed sensitivity that was at least one log greater when using the enhanced detector particles as compared to the current detector particles.

In a multiple analyte system, increasing the size of the particle also increases the intensity of the signal generated in the antigen/antibody response, thereby increasing the sensitivity. This is surprising because the amount of gold in the capture area is limiting, as a practical matter. Thus, more numerous smaller particles can be used, and the smaller particles have a larger surface to volume ratio. Without wishing to be bound by theory, this phenomenon appears to occur because there are more antibodies per particle, thereby allowing greater avidity for an antigen. In these experiments, the gold particle, the immobilization method, and the conditions of binding the antibodies to the surface were evaluated. The result of these studies was successful immobilization of approximately four times more antibodies per gold particle, which yielded a substantially enhanced detector particle.

In summary, we have demonstrated increased signal intensity across multiple bacterial species by using larger, darker gold particles, resulting in improved sensitivity and accuracy with easier to read results.

### Example 3

### Synthesis of a Pan-Generic Reagent Particle

Rabbit IgG is diluted to desired concentrations in 2-fold concentrated binding buffer. Those of skill in the art will appreciate that, any binding buffer suitable for binding IgG to Protein A can be used. Typical concentrations for coupling range from 0.1 to 1 μg/ml*OD of gold. If 5 ml of gold colloid at OD555=10 is to be coupled with a ratio of 0.1 ug/ml*OD, then IgG is diluted to a concentration of 1.0 ug/ml in a volume of 5 ml. Diluted antibody is mixed with an equal volume of 80 nm gold particles coated with protein A (sPA) concentrated to twice the desired final desired concentration of particles. Incubation is for a minimum of one hour before testing, but overnight incubation also could be advantageous.

What is claimed is:

1. A detectable particle-bound binding agent-based device for detecting analytes in a multi-analyte sample utilizing two or more populations of detectable particles, wherein a first population of detectable particles are particles from about 20 nm to about 60 nm in diameter and a second population of detectable particles are particles from greater than about 60 nm to about 120 nm in diameter, wherein the first population of detectable particles are bound to one or more binding agents having specificity for a first population of analytes and the second population of detectable particles are bound to one or more binding agents having specificity for a second population of analytes.

2. The device according to claim 1, wherein the first population and second population of analytes are the same.

3. The device according to claim 1, wherein the first population and second population of analytes are different from each other.

4. The device according to claim 3, wherein analytes of the first population of analytes are present in the sample at a higher concentration than analytes of the second population of analytes.

5. The device according to claim 1, wherein the detectable particles are made of a material selected from gold, silver and platinum.

6. The device according to claim 5, wherein the detectable particles are made of gold.

7. The device according to claim 1, wherein the analytes are bacterial antigens.

8. The device according to claim 1, wherein the binding agents are bound to the detectable particles via a linker.

9. The device according to claim 8, wherein the linker is selected from the group consisting of protein A, protein G, protein L, and a species-specific anti-immunoglobulin antibody.

10. The device according to claim 9, wherein the linker is protein A.

11. The device according to claim 8, wherein the linker is present on the detectable particles at a surface density of from about 1 to about 10 molecules of linker per 100 nm².

12. The device according to claim 1, wherein the one or more binding agents bound to the first population of detectable particles comprises one or more antibodies.

13. The device according to claim 12, wherein the antibodies are monoclonal antibodies.

14. The device according to claim 1, wherein the one or more binding agents bound to the second population of detectable particles comprises one or more antibodies.

15. The device according to claim 14, wherein the antibodies are polyclonal antibodies

16. The device according to claim 1, wherein the binding agents are selected from polyclonal antibodies, monoclonal antibodies, functional fragments thereof, and combinations thereof.

17. The device according to claim 16, wherein the polyclonal antibodies or monoclonal antibodies comprise one or more pan-generic antibodies.

18. A lateral flow device for detecting bacteria in a sample, the device comprising a flow path for the sample and further comprising a binding agent that specifically binds a bacterial antigen, wherein the binding agent is immobilized on two or more populations of detectable particles wherein a first population of particles comprises particles from about 20 nm to about 60 nm in diameter and a second population of particles comprises particles from greater than about 60 nm to about 120 nm in diameter, wherein the first population of particles is bound to one or more binding agents having specificity for a first population of analytes and the second population of particles is bound to one or more binding agents having specificity for a second population of analytes; and a capture binding agent that captures the one or more population of particles, wherein the capture binding agent is immobilized on the flow path, and wherein the population of detectable particles is disposed along the flow path such that the sample contacts the population of detectable particles before contacting the capture binding agent.

19. The device according to claim 18, wherein the first and second population of analytes are the same.

20. The device according to claim 18, wherein the first and second population of analytes are different from each other.

21. The device according to claim 18, wherein the binding agents are bound to the detectable particles via a linker.

22. The device according to claim 21, wherein the linker is selected from the group consisting of protein A, protein G, protein L, and a species-specific anti-immunoglobulin antibody.

23. The device according to claim 22, wherein the linker is protein A.

24. The device according to claim 21, wherein the linker is present on the detectable particles at a surface density of from about 1 to about 10 molecules of protein A per 100 nm².

25. The device according to claim 18, wherein the first population of detectable particles are about 40 nm in diameter and the second population of detectable particles are about 80 nm in diameter.

26. A method for detecting analytes in a sample, comprising contacting the sample with one or more binding agents specific for one or more analytes, wherein the one or more binding agents is immobilized on two or more population of detectable particles, wherein a first population of detectable particles comprises particles from about 20 nm to about 60 nm in diameter and a second population of detectable particles comprises particles from greater than about 60 nm to about 120 nm in diameter, wherein the first population of detectable particles is bound to a population of one or more binding agents having specificity for a first population of analytes and the second population of detectable particles is bound to a population of one or more binding agents having specificity for a second population of analytes; and wherein the sample is contacted with the one or more binding agents under conditions that permit binding between the binding agent and the analyte, wherein binding of the binding agent by the analyte indicates the presence of analyte in the sample.

27. The method according to claim 26, wherein the first and second population of analytes are the same.

28. The method according to claim 26, wherein the first and second population of analytes are different from each other.

29. The method according to claim 26, wherein the one or more binding agents comprises one or more pan-generic binding agents.

30. The method according to claim 26, further comprising contacting an immobilized capture binding agent with the detectable particle under conditions that permit binding between the immobilized capture binding agent and the detectable particle with the binding agent, wherein binding of the detectable particle by the capture binding agent indicates the presence of analyte in the sample.

31. The method according to claim 26, wherein analytes of the first population of analytes are present in the sample at a higher concentration than analytes of the second population of analytes.

32. The method according to claim 26, wherein the detectable particles are made of a material selected from gold, silver and platinum.

33. The method according to claim 32, wherein the detectable particles are made of gold.

34. The method according to claim 26, wherein the analytes are bacterial antigens.

35. The method according to claim 26, wherein the binding agents are bound to the detectable particles via a linker.

36. The method according to claim 35, wherein the linker is selected from the group consisting of protein A, protein G, protein L, and a species-specific anti-immunoglobulin antibody.

37. The method according to claim 36, wherein the linker is protein A.

38. The method according to claim 35, wherein the linker is present on the detectable particles at a surface density of from about 1 to about 10 molecules of linker per 100 nm².

39. The method according to claim 26, wherein the binding agents are selected from polyclonal antibodies, monoclonal antibodies, functional fragments thereof, and combinations thereof.

40. The method according to claim 39, wherein the polyclonal antibodies or monoclonal antibodies comprise one or more pan-generic antibodies or functional fragments thereof.

41. A method for increasing specificity and/or sensitivity in a binding assay to detect binding of a plurality of analytes in a multi-analyte sample to a particle-bound binding agent, comprising contacting the sample with one or more binding agents specific for one or more analytes, wherein the one or more binding agents is immobilized on two or more populations of detectable particles, wherein a first population of detectable particles comprises particles from about 20 nm to about 60 nm in diameter and a second population of detectable particles comprises particles from greater than about 60 nm to about 120 nm in diameter, wherein the first population of detectable particles is bound to one or more binding agents having specificity for a first population of analytes and the second population of detectable particles is bound to one or more binding agents having specificity for a second population of analytes; wherein the sample is contacted with the one or more binding agents under conditions that permit binding between the binding agent and the analyte and binding of the binding agent by the analyte indicates the presence of analyte in the sample; and wherein the increase in sensitivity and/or specificity is in comparison to the same method but with a first population of detectable particles and a second population of detectable particles that have the same diameter.

42. The method according to claim 41, wherein the first and second population of analytes are the same.

43. The method according to claim 41, wherein the first and second population of analytes are different from each other.

44. The method according to claim 41, wherein the one or more binding agents comprises one or more pan-generic binding agents.

45. The method according to claim 41, further comprising contacting an immobilized capture binding agent with the detectable particle under conditions that permit binding between the immobilized capture binding agent and the detectable particle with the binding agent, wherein binding of the detectable particle by the capture binding agent indicates the presence of analyte in the sample.

46. The method according to claim 41, wherein analytes of the first population of analytes are present in the sample at a higher concentration than analytes of the second population of analytes.

47. The method according to claim 41, wherein the detectable particles are made of a material selected from gold, silver and platinum.

48. The method according to claim 47, wherein the detectable particles are made of gold.

49. The method according to claim 41, wherein the analytes are bacterial antigens.

50. The method according to claim 41, wherein the binding agents are bound to the detectable particles via a linker.

51. The method according to claim 50, wherein the linker is selected from the group consisting of protein A, protein G, protein L, and a species-specific anti-immunoglobulin antibody.

52. The method according to claim 51, wherein the linker is protein A.

53. The method according to claim 50, wherein the linker is present on the detectable particles at a surface density of from about 1 to about 10 molecules of linker per 100 nm².

54. The method according to claim 41, wherein the binding agents are selected from polyclonal antibodies, monoclonal antibodies, functional fragments thereof, and combinations thereof.

55. The method according to claim 54, wherein the polyclonal antibodies or monoclonal antibodies comprise one or more pan-generic antibodies or functional fragments thereof.

56. The method of claim 26 or 41, further comprising treating the sample with a mixture of enzymes to increase the sensitivity of the assay.

57. The method of claim 56, wherein the mixture of enzymes comprises one or more bacterial cell wall-specific endopeptidase and one or more bacterial cell wall-specific glycoside hydrolase.

58. The method of claim 57, wherein the bacterial cell wall-specific endopeptidase is lysostaphin.

59. The method of claim 57, wherein the bacterial cell wall-specific glycoside hydrolase is lysozyme.

60. The method of claim 57, wherein the bacterial cell wall-specific endopeptidase is lysostaphin and the bacterial cell wall-specific glycoside hydrolase is lysozyme.

61. The method of claim 57, wherein the bacterial cell wall-specific endopeptidase is present in a concentration of from about 0.06 mg/ml to about 200 mg/ml.

62. The method of claim 57, wherein the bacterial cell wall-specific glycoside hydrolase is present in a concentration of from about 0.0025 mg/ml to about 15 mg/ml.

63. The method of claim 58, wherein the lysostaphin is present in a concentration of from about 0.06 mg/ml to about 200 mg/ml.

64. The method of claim 59, wherein the lysozyme is present in a concentration of from about 0.0025 mg/ml to about 15 mg/ml.

65. The method of claim 56, wherein the enzyme treatment further comprises a surfactant.

66. The method of claim 56, wherein treating of the sample is carried out at room temperature.

67. The method of claim 56, wherein the enzymes are selected from the group consisting of lysostaphin, lysozyme, lipase, mutanolysin, labiase, achromopeptidase, trypsin, proteinase K, an autolysin, bacteriophage-encoded lytic enzymes, and combinations thereof.

68. A binding assay for detecting the presence of bacteria from a plurality of genera in a sample comprising the step of treating the sample with enzymes to increase the sensitivity of the binding assay.

69. The assay of claim 68, wherein the sample is selected from the group consisting of: urine, sputum, spinal fluid, ascites, blood, lavage fluid, dialysis fluid and blood products.

70. The assay of claim 68, wherein the pharmaceutical composition is a blood product selected from the group consisting of: whole blood, leukocytes, hematopoietic stem cells, platelets, red blood cells, plasma, bone marrow and serum.

71. The assay of claim 68, wherein the treatment is with two or more enzymes and wherein at least one enzyme is a bacterial cell wall-specific endopeptidase and wherein at least one enzyme is a bacterial cell wall-specific glycoside hydrolase.

72. The assay of claim 71, wherein the endopeptidase is selected from the group consisting of: lysostaphin and achromopeptidase.

73. The assay of claim 72, wherein the endopeptidase is lysostaphin.

74. The assay of claim 71, wherein the glycoside hydrolase is selected from the group consisting of: lysozyme and mutanolysin.

75. The assay of claim 74, wherein the glycoside hydrolase is lysozyme.

76. The assay of claim 71, wherein the two or more enzymes comprise lysostaphin and lysozyme.

77. The assay of claim 71 or 72, wherein the bacterial cell wall-specific endopeptidase is present in a concentration of from about 0.06 mg/ml to about 200 mg/ml.

78. The assay of claim 71, wherein the bacterial cell wall-specific glycoside hydrolase is present in a concentration of from about 0.0025 mg/ml to about 15 mg/ml.

79. The assay of claim 73 or 76, wherein the lysostaphin is present in a concentration of from about 0.06 mg/ml to about 200 mg/ml.

80. The assay of claim 75 or 76, wherein the lysozyme is present in a concentration of from about 0.0025 mg/ml to about 15 mg/ml.

81. The assay of claim 68, wherein the treatment further comprises a surfactant.

82. The assay of claim 68, wherein treating the sample is carried out at room temperature.

83. The assay of claim 68, wherein the enzymes are selected from the group consisting of lysostaphin, lysozyme, mutanolysin, labiase, achromopeptidase, trypsin, proteinase K, an autolysin, bacteriophage-encoded lytic enzymes, and combinations thereof.

84. The assay of claim 68, wherein the sample is contacted with the enzymes prior to contacting the sample with a binding reagent.

85. The assay of claim 68, wherein the sample is contacted with the enzymes and a binding reagent at the same time.

86. The assay of claim 84 or 85, wherein the sample is incubated with the enzymes prior to detecting the presence or absence of binding.

87. The assay of claim 29 or 30, wherein the presence or absence of binding is detected directly after contacting the sample with the binding agent.
